(19) United States
(12) Patent Application Publication
(10) Pub. No.: US 2015/0136138 A1
(43) Pub. Date: May 21, 2015

(54) HEADGEAR ASSEMBLY WITH IMPROVED STABIIZATION

(71) Applicant: KONINKLIJKE PHILIPS N.V., EINDHOVEN (NL)

(72) Inventors: Sander Theodoor Pastoor, Eindhoven (NL); Krijn Frederik Bustraan, Eindhoven (NL); Timon Rutger Grob, Eindhoven (NL); Tom Jan Severijn, Eindhoven (NL)

(73) Assignee: KONINKLIJKE PHILIPS N.V., Eindhoven (NL)

(21) Appl. No.: 14/369,817

(22) PCT Filed: Dec. 27, 2012
(86) PCT No.: PCT/IB2012/057737

§ 371 Date: Jun. 30, 2014

Related U.S. Application Data
(60) Provisional application No. 61/582,502, filed on Jan. 3, 2012.

(51) Int. Cl. A61M 16/06

(52) U.S. Cl. CPC A61M 16/0694; A61M 16/0616; A61M 16/0666
USPC 128/205.25

(57) ABSTRACT

A headgear assembly is disclosed for use in a patient interface assembly adapted to communicate a flow of gas with an airway of a patient comprising: at least a first headgear portion (9, 11) disposed during use on a first side of a patient's face in an area close to a first eye of the patient; a first stabilization member (2) operatively coupled to the first headgear portion so as to limit the movement of the headgear portion in a direction towards the first eye; and a patient interface assembly comprising such a headgear assembly.

### FIELD OF THE INVENTION

The present invention relates to a patient interface assembly for being connected to a patient's respiratory system in order to provide a flow of gas to an airway of the patient and, in particular, to a patient interface assembly that includes head gear, for instance comprising cheek mount supports, to properly locate and fix the patient interface assembly on the face of the user.

### BACKGROUND OF THE INVENTION

There are numerous situations where it is necessary or desirable to deliver a flow of breathing gas non-invasively to the airway of a patient, i.e., without intubating the patient or surgically inserting a tracheal tube in the esophagus. For example, it is known to ventilate a patient using a technique known as non-invasive ventilation. It is also known to deliver continuous positive airway pressure (CPAP) or variable airway pressure, such as a bi-level pressure that varies with the patient's respiratory cycle or an auto-titrating pressure that varies with the monitored condition of the patient, to the airway of a patient/user. Typical pressure support therapies are provided to treat a medical disorder, such as sleep apnea syndrome, in particular, obstructive sleep apnea (OSA) or congestive heart failure and/or other medical and respiratory disorders, such as Cheynes-Stokes respiration, congestive heart failure, and stroke.

Non-invasive ventilation and pressure support therapies involve the placement of a patient interface assembly, which is typically a nasal or nasal/oral mask, on the face of a patient to interface the ventilator or pressure support system with the airway of the patient so that a flow of breathing gas can be delivered from the pressure/flow generating patient interface assembly to the airway of the patient. It is known to maintain such masks on the face of a patient by a headgear having upper and lower straps, each having opposite ends threaded through connecting elements provided on the opposite sides and top of a mask.

Because such masks are typically worn for an extended period of time, a variety of concerns must be taken into consideration. For example, in providing a pressure support therapy to treat OSA, the patient normally wears the patient interface assembly all night long while he or she sleeps. Patient interface development has generally involved balancing of two competing goals: a) secure attachment to and seal with the user's face to create an airtight seal in order to facilitate the required positive airway pressure, and b) comfort to the user in order to maximize patient compliance, i.e., usage of the medical therapy. An airtight seal can be achieved by tightening the mask down firmly against the patient's face. However, this solution oftentimes results in discomfort to the user due to relatively high strapping forces needed to ensure a secure seal against the patient and less than satisfactory patient compliance. Alternatively, the mask may be fit loosely on the patient's face to enhance comfort. However, the effectiveness of the mask may be compromised if it is too loose.

In WO 2011/110961, a patient interface assembly is described that includes a support member comprising a central support portion and a pair of cheek mount supports coupled to the central portion. Each cheek mount support is configured to apply a force a side a user's cheekbone while applying substantially no force over an apex of such a user's cheekbone responsive to the patient interface assembly being worn by a user. A seal member is coupled to the support portion. The seal member is adapted to seal against a surface of a user to communicate a flow of gas with an airway of such a user. A conduit coupling member is coupled to the seal member. The seal member comprises two conical nasal prongs, adapted for being inserted in the nasal passages of the patient.

The stability and fixation of the nasal prongs is obtained by the central support which is connected with cheek mount supports which are in contact with the face of the patient in the area around the cheekbone.

The stability of the seal member and, if present, nasal prongs depends at least partially on the stability of headgear assembly, and thus also on the stability of the cheek mount supports. The stability of the headgear, especially of the cheek mount supports is thus crucial. Moreover, part of the cheek mount support is positioned near the eye of the patient, such that a translation of the cheek mount support towards the eye will right away disturb the user as this will cause at first instance pressure on and deformation of the skin tissue around the eye, pushing the skin tissue into the eye. In worst case the cheek support may also shift into the eye, with is off course unacceptable. There exists a need for further improvements for stabilizing the cheek mount supports in industry.

### SUMMARY OF THE INVENTION

Accordingly, it is an object of the present invention to provide a patient interface assembly that overcomes the shortcomings of the conventional patient interface assembly. This object is achieved according to embodiments of the present invention by providing a patient interface assembly that includes an additional headgear stabilizer, for instance a cheek mount support stabilizer member or means, the additional cheek mount support stabilizer member being configured to further limit the freedom of movement of a portion of the headgear, for instance of the cheek mount supports, without substantially impacting the comfort of the patient when using and wearing the patient interface assembly.

Particular and preferred aspects of the invention are set out in the accompanying independent and dependent claims. Features from the dependent claims may be combined with features of the independent claims and with features of other dependent claims as appropriate and not merely as explicitly set out in the claims.

In a first aspect of the present invention, a headgear assembly is disclosed for use in a patient interface assembly adapted to communicate a flow of gas with an airway of a patient, the headgear assembly comprising:
at least a first headgear portion disposed during use on a first side of a patient's face in an area close to a first eye of the patient;
a first stabilization member operatively coupled to the first headgear portion so as to limit the movement of the headgear portion in a direction towards the first eye.

The area close to the first eye of the patient can comprise the cheekbone area. It can moreover comprise the area between the cheek bone and the lower rim or lower eyelid of the respective eye. The lower boundary of the area close to the eye can be an area defined by a distance from 0 to 10 cm, more preferable between 0.5 to 8 cm, more preferably between 1 to 6 cm from the lower rim of the eye, defined as the skin fold at the bottom of in the lower eyelid of the eye. The distance is not larger than the distance between the apex of the cheekbone and the eye.

The movement of the first headgear portion towards the eye, which is limited according to aspects of the present invention, may otherwise have brought the first headgear portion from a first position with respect to the eye or first distance from the eye that would be experienced as comfortable to the patient, to a second position with respect to the eye or second distance from the eye that would be experienced as uncomfortable to the patient. The second position or distance corresponding to discomfort, could for instance be a position or distance wherein a substantial direct or indirect pressure on the lower eyelid or on the eye would be induced by said first headgear portion.

According to preferred embodiments, the first stabilization member is adapted and/or arranged for applying a force on the first headgear portion in a direction away from the user's first eye during use.

According to preferred embodiments, the first stabilization member is adapted and/or arranged for restricting a movement of the first headgear portion such that the first headgear portion cannot move in a direction towards the user's first eye during use.

According to preferred embodiments, the headgear further comprises a second headgear portion disposed during use on a second side of a patient's face in an area close to a second eye of the patient and a second stabilization member operatively coupled to the second headgear portion so as to limit the movement of the headgear portion in a direction towards the second eye.

The characteristics described above for the first headgear portion are also applicable for the second headgear portion. According to preferred embodiments, a headgear has a left-right symmetry. Preferably the configuration and arrangement of the first (left) headgear portion and the second (right) headgear portion is symmetrical.

According to preferred embodiments, the first stabilization member and the second stabilization member are connected at a first end to the first headgear portion and the second headgear portion respectively, and are connected at a second end to each other, thereby forming a single headgear strap connecting the first headgear portion and the second headgear portion.

According to preferred embodiments, the single headgear strap comprises at least one length adjustment means for adjusting the length of the single headgear strap. By adjusting the length of the strap, the strap can for instance be tightened to or released from the head of the patient.

According to preferred embodiments, the single headgear strap comprises a first and a second length adjustment means, a first length adjustment means being adapted for being positioned at a first side of the user's face during use, and a second length adjustment means being adapted for being positioned at a second side of the user's face during use. This can provide the advantage that a patient may tighten the strap one or both sides of the patient's face, and may have the option to choose in function of the circumstances. Moreover, by having two length adjustment means being arranged symmetrically with respect to the user's face, forces on the left and right side of the face can be balanced. In certain embodiments, for instance when the single strap would pass under or over the chin of the patient, see further, it may also reduce the need of gliding of the strap over the skin in the chin area when tightening the strap. Indeed, both sides of the single strap can be for instance shortened sequentially or contemporarily, while a strap portion positioned over the chin does not have to change position when tightening the single strap. Therefor, for instance, a high friction material may be provided in the region of the single strap which is adapted and/or arranged for being positioned in the chin area of the patient, e.g. under or over the chin. These embodiments and advantages may similarly apply when the single strap is positioned differently, as will be described in more detail below.

According to preferred embodiments of the present invention, the first headgear portion and, if present, the second headgear portion comprise a cheek mount support.

According to preferred embodiments of the present invention, the single headgear strap is adapted for being arranged such that it passes over the back of a head of the user.

According to preferred embodiments of the present invention, the single headgear strap is adapted for being arranged such that it passes over the back of a head of the user and above the ears of the user when the patient interface assembly is worn by the user.

According to preferred embodiments of the present invention, the single headgear strap is adapted for being arranged such that it passes over the back of a head of the user and over the ears of the user when the patient interface assembly is worn by the user.

According to preferred embodiments of the present invention, the single headgear strap is adapted for being arranged such that it passes over the back of a head of the user and under the ears of the user when the patient interface assembly is worn by the user.

According to preferred embodiments of the present invention, the single headgear strap is adapted for being arranged such that it passes over the back of a head of the user and around the ears of the user when the patient interface assembly is worn by the user.

According to preferred embodiments of the present invention, the openings are adapted for allowing ears of the patient to pass through. Preferably each of the pair of openings is adapted for closely fitting around a basis of the ears of the user.

According to preferred embodiments of the present invention, the single headgear strap is adapted for being arranged such that it passes under and/or over a chin of the user when the patient interface assembly is worn by the user.

According to preferred embodiments of the present invention, the cheek mount supports are rigid or semi rigid.

According to preferred embodiments of the present invention, each of the cheek mount support comprises a first member that is adapted to pass between an apex of a cheekbone and a eye of a user when the patient interface assembly is being worn by the patient; and

a second member that is adapted to pass below the apex of the cheekbone when the patient interface assembly is being worn by the patient; and the first/second stabilization member(s) are connected to the respective first members.

According to preferred embodiments of the present invention, the first member and the second member of a cheek mount support are joined at each end to define a loop-shaped structure, and the looped-shaped structure is sized and configured to fully encircle an apex of a cheek bone of the patient when the patient interface assembly is being worn by such the patient.

According to preferred embodiments of the present invention, the cheek mount supports comprise a first end portion positioned towards a central part of the user's face and a second end portion positioned further away from the central part, when the patient interface assembly is worn by the user, and the stabilization member (cheek mount support stabilization member) is connected to the first end portions.

This can for instance be advantageous in the case with the cheek mount support stabilization member is passing over and/or under the chin, as the length of the strap can be reduced without affecting the functionality of the strap. This can reduce cost and increase comfort to the user during wear of the patient interface assembly.

According to preferred embodiments of the present invention, the cheek mount supports comprise a first end portion positioned towards a central part of the user's face and a second end portion positioned further away from the central part, when the patient interface assembly is worn by the user, and the stabilization member (cheek mount support stabilization member) is connected to the second end portions.

This can for instance be advantageous in the case with the cheek mount support stabilization member is passing over the back of the head, as for instance above, under, over, or around the ears or the user. The length of the strap can be reduced without affecting the functionality of the strap. This can reduce cost and increase comfort to the user during wear of the patient interface assembly.

According to preferred embodiments of the present invention, the stabilization member is at least partially stretchable. For instance, the single stabilization strap can comprise a stretchable portion near its center portion. The center portion can for instance be the portion corresponding to the chin area of the patient. According to other preferred embodiments, the single strap can fully consist of a stretchable material.

According to a second aspect of the present invention, a patient interface assembly for communicating a flow of gas with an airway of a patient is disclosed comprising
a headgear assembly according to any of the embodiments of the first aspect;
a seal member operatively coupled to the headgear assembly, wherein the seal member is adapted to seal against a surface of the user to communicate a flow of gas with an airway of the user when using the patient interface assembly.

According to preferred embodiments of the present invention, the seal member comprises a pair of nasal prongs, the nasal prongs being adapted for being inserted in nasal passages of the user during use.

According to embodiments of the second aspect of the present invention, a patient interface assembly is disclosed, comprising:
a support member comprising a central support portion and a first and a second cheek mount support coupled to the central portion, wherein each the cheek mount support is configured to apply a force on or a side a user's cheekbone and has a first freedom of movement when the patient interface assembly is worn by the user;
a seal member operatively coupled to the support portion, the seal member for instance comprising a pair of nasal prongs, wherein the seal member is adapted to seal against a surface of the user to communicate a flow of gas with an airway of the user, for instance by means of the nasal prongs being inserted in nasal passages of the user; and
an additional cheek mount support stabilization member, the cheek mount support stabilization member being adapted and arranged in order to limit the first freedom of movement of the cheek mount supports respective to the user's cheek bone when the patient interface assembly is being worn by such a user.

According to preferred embodiments of the present invention, the patient interface assembly further comprises a head strap or head gear assembly directly coupled to the cheek mount supports, or in another view, the cheek mount supports are being part of a headgear assembly. The use of such a head strap or head gear assembly alone, without the additional cheek mount stabilization member, may correspond to the first freedom of movement. The additional use of the cheek mount stabilization member may result in a second freedom of movement, which is more limited than the first freedom of movement.

According to preferred embodiments, the additional cheek mount support stabilization means is adapted and arranged in order to limit or block a movement towards the eye of the cheek mount supports when in use. The cheek mount support stabilization member can also be adapted and arranged to limit or block a rotational movement of the cheek mount support.

According to preferred embodiments, the cheek mount stabilization member comprises a strap-like element which is connected at a first end to the first cheek mount support and at a second end to the second cheek mount support and which is adapted for passing over the back of a head of the user when the patient interface assembly is worn by the user.

The cheek mount stabilization member is thereby preferably independent from, or not directly coupled with the head strap or head gear corresponding with the first freedom of movement of the patient interface assembly.

According to preferred embodiments, the cheek mount support stabilization member is adapted for being arranged such that it passes above the ears of the user when the patient interface assembly is worn by the user.

According to preferred embodiments, the cheek mount support stabilization member is adapted for being arranged such that it passes over the ears of the user when the patient interface assembly is worn by the user.

According to preferred embodiments, the cheek mount support stabilization member is adapted for being arranged such that it passes under the ears of the user when the patient interface assembly is worn by the user.

According to preferred embodiments, the cheek mount support stabilization member is adapted for being arranged such that it passes around the ears of the user when the patient interface assembly is worn by the user. The cheek mount support stabilization member can therefor for instance comprise a pair or openings, the openings being adapted for allowing ears of the patient to pass through. According to preferred embodiments, each of the pair of openings is adapted for closely fitting around a basis of the ears of the user.

According to preferred embodiments, the cheek mount stabilization member comprises a strap-like element which is connected at a first end to the first cheek mount support and at a second end to the second cheek mount support, and which is adapted for being arranged such that it passes under and/or over a chin of the user when the patient interface assembly is worn by the user.

According to preferred embodiments, the cheek mount supports are rigid. They can for instance be formed from a material consisting of metal, plastic, or a combination thereof.

According to preferred embodiments, the cheek mount stabilization member is at least partially stretchable.

The cheek mount supports can be as described for the first aspect of the present invention.

The above and other characteristics, features and advantages of the present invention will become apparent from the following detailed description, taken in conjunction with the accompanying drawings, which illustrate, by way of example, the principles of the invention. This description is given for the sake of example only, without limiting the scope of the invention. The reference figures quoted below refer to the attached drawings.

### BRIEF DESCRIPTION OF THE DRAWINGS

FIG. 1 is a perspective view of a patient interface assembly according to the prior art, on which embodiment according to the present invention can be based.

FIG. 2 is a perspective view of a human skull illustrating the cheekbones and other shape characteristics of a human skull, which indicate the state of the art position of cheek mount supports.

FIG. 3 is a perspective view of a first preferred embodiment according to aspects of the present invention.

FIG. 4 is a perspective view of a second preferred embodiment according to aspects of the present invention.

FIG. 5 is a perspective view of a third preferred embodiment according to aspects of the present invention.

FIG. 6 is a perspective view of a fourth preferred embodiment according to aspects of the present invention.

FIG. 7 is a perspective view of a fifth preferred embodiment according to aspects of the present invention.

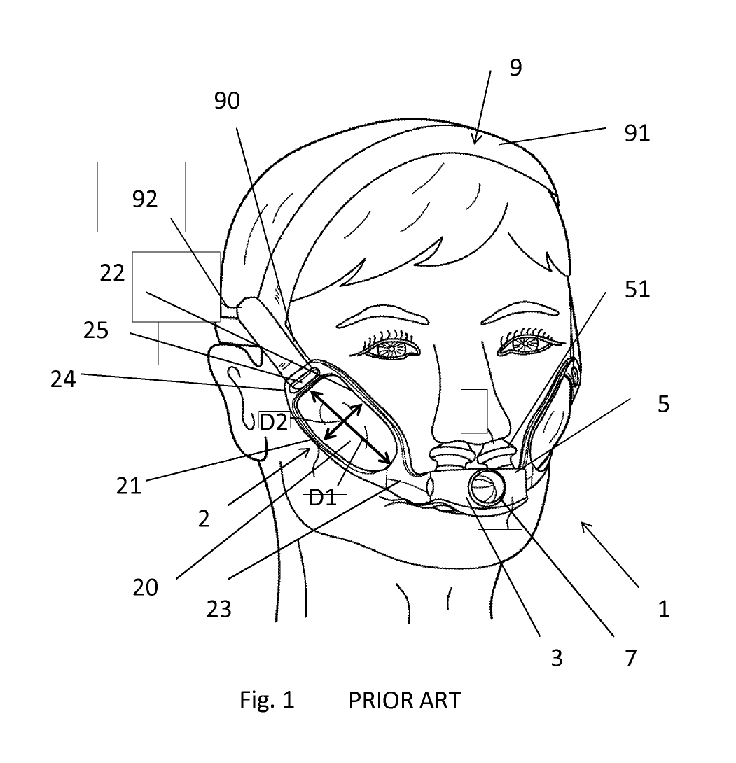

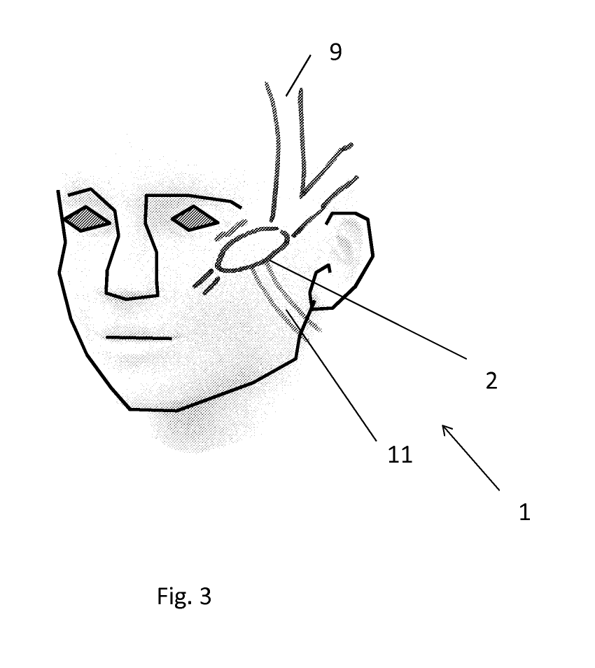

In FIGS. 3 to 7 the patient interface assembly as depicted in FIG. 1, or any of its state of the art alternatives, is represented only schematically.

### DETAILED DESCRIPTION OF THE EMBODIMENTS

As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

FIG. 1 illustrates a prior art patient interface assembly according to WO 2011/110961 being positioned on the face of a user or patient, which can be the basis for the improvements embodied in the embodiments of the present invention. The patient interface device 1 comprises two cheek mount supports 2 (one on the left and one on the right side of the face), which are coupled to each other via a central portion 3. A seal member 5 is coupled to the central portion 3. The seal member 5 is adapted to seal against a surface of a user to communicate a flow of gas with an airway of such a user. The seal member 5 therefore comprises a pair of nasal prongs 51 which are adapted for being inserted into the patient's nasal passages and to hereby make a substantially or completely airtight seal with the internal surface of the nasal passages of the patient. Further, typically a conduit coupling member 7 is coupled to the seal member 5. The conduit coupling member is connected with a conduit or circuit (not depicted), which connects the patient interface assembly with a pressure support system (not depicted). The seal member 5 comprises internal channels which distribute the inflow or outflow of gas to appropriate locations. For instance, internal channels can be provided which distribute the inflow of gas towards the nasal prongs 51.

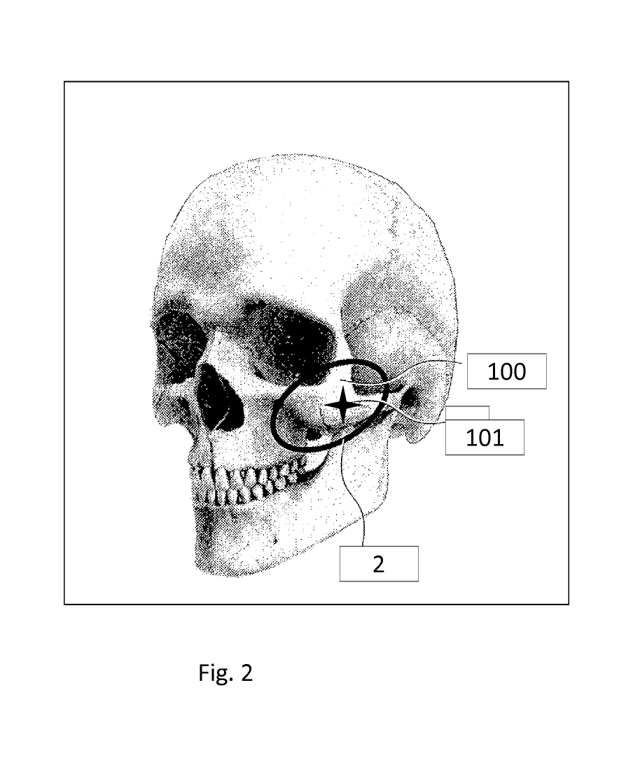

Each cheek mount support 2 includes a first member 21 and a second member 22, which, in the illustrated embodiment, are coupled at first end portion 23 and second end portion 24 of the cheek support member. In this manner, each cheek mount support has a loop-shaped structure having an opening 20 defined between the first and second members. Each cheek support member 2, and, in particular, the first and second members and their interconnections that define the loop-shaped structure, are preferably sized and configured such that when the patient interface assembly 1 is worn by the user, the loop-shaped member is configured to be disposed over opposing sides a user's cheekbone while not being disposed over an apex 101 of such a user's cheekbone. Stated another way, illustrated in FIG. 2, the zygomatic bone 100 (also called cheekbone) has a peak portion 101, i.e., a portion that protrudes the furthest from the face. Preferably no part of cheek mount support applies a force against peak portion 101 of the cheekbone. Instead, the peak portion of the cheek bone protrudes through opening 20 of cheek support mount 2 so that first member 21 and second member 22 rest on either side of the cheekbone. In the illustrated embodiment, cheek support member 2 fully encircles, but does not pass over the apex of, a user's cheek area. Each cheek mount support is configured to apply a force a side a user's cheekbone while applying substantially no force over an apex of such a user's cheekbone when the patient interface assembly is being worn by a user.

The spacing between first member 21 and second member 22, i.e., the width of opening 20, as indicated by dimension D2 in FIG. 2B, can for instance range from 1 to 8 cm, or from 2 cm to 6 cm. The spacing between first end portion 23 and the second end portion 24, i.e., the length of opening 20, as indicated by dimension D1 in FIG. 2B can for instance range from 1 cm to 12 cm or from 2 to 10 cm. Smaller and larger dimensions are also not excluded. This range of dimensions ensures that the components of cheek mount support 2 remain over the sides of the cheekbone but are not disposed over the apex of the cheekbone. As a result, the cheekbone and overlying tissue becomes a support structure for holding the patient interface assembly in place on the face. Using opening 20 to “capture” or “anchor” to the cheekbone also provides for easy alignment of support member on the user's face and enhances the stability of the support member of the face, and thereby decreasing leaks and patient discomfort.

Although probably less comfortable in wear and/or intrinsically less stable, alternatively, other state of the art cheek mount supports can be used instead. This can be for instance cheek mount supports which do pass over the apex of the user, as for instance the cheek mount supports as described above which additionally pass over the apex of the user, thereby possibly exerting a pressure on the cheekbone of the user. Or cheek mount supports which are positioned with respect to the cheek bone by exerting a pressure on the cheek bone area, and not around the cheek bone area. These types of patient interface assemblies can also be improved according to aspects of the present invention.

The cheek mount supports 2 comprise a coupling member 25 (e.g. an opening or similar means) near their second end portion 24 for coupling with a head strap or head gear 9 (in another view, the headgear can comprise the cheek mount support). Such a head gear 9 can comprise on both sides of the patient's head, a first strap 90 which is connected to the cheek mount support 2 by means of the coupling member 25 on one end and which bifurcates into two further straps 91 and 92, the respective further straps of both cheek support members being connected to each other. The strap formed by the first straps 91 may for instance pass over the top of the head of the user, while the strap formed by the second straps 92 may for instance pass over the back of the head. Although the above described configuration has proven to be good, it will be appreciated by the skilled person that also other head strap or head gear combinations are possible.

In FIG. 2, a human skull is depicted. The skull comprises a cheek bone 100 (also zygomatic bone) and the position of a cheek mount support 2 of the patient interface assembly 1 with respect to the skull is illustrated, as it is positioned in the prior art. The cheek bone comprises an apex 101, being a peak portion which is a portion protruding the furthest from the face (approximately indicated throughout the figures with a “star”). The cheek mount support is positioned with respect to the cheekbone by applying a force a side the cheekbone.

Embodiments according to aspects of the present invention, disclose patient interface assemblies as described in any of the above examples, and do further comprise a cheek mount support stabilizer means 4.

The cheek mount supports 2 cooperate with the cheek mount stabilizer means 4, thereby avoiding or reducing the freedom of movement of the cheek mount supports 2, by further limiting the translational and/or rotational movement of the cheek mount supports 2.

A movement of the cheek mount support towards the eye would burden the patient. Moreover any translational or rotational movement of the cheek mount supports may result in exerting of forces on the internal of the nose, and may be experienced as still very uncomfortable to the user and as such a movement may break or change the properties of the airtight seal between the nasal prongs and the internal surface of the nasal passages of the patient.

The cheek mount support stabilizer means 4 is being configured to apply a stabilizing force on the cheek mount support responsive to the patient interface assembly being worn by the user, and thereby limits, reduces or avoids the presence of additional forces which would otherwise be acting on the user's nose, especially on the nasal passages of the use, during use of the patient interface assembly.

The cheek support stabilisation members according to embodiments of the present invention can be straps.

In the FIGS. 3 to 7, the patient interface assembly can be based on any of its state of the art alternatives as described above in relation with FIG. 1, and is represented only schematically.

FIG. 3 illustrates a patient interface assembly 1 according to a first embodiment, when positioned on the face of a user or patient. The patient interface assembly is as depicted and described in relation with FIG. 1, but comprises further a cheek support stabilization member which is directly coupled with a first end to a first cheek mount support and with a second end to a second cheek mount support. The cheek support stabilization member is passing over the back of the head and under the ears of the user, and can be tightened, for instance on both sides to the cheek bone stabilizer members 2. The connection can be for instance with Velcro (hook and loop fastener) on the strap or with clips. The advantage is that this stabilization strap member creates a pull down force in a direction away from the eye. Therefore the chance that the cheek bone stabilizer member 2 will slide towards the eyes is limited.

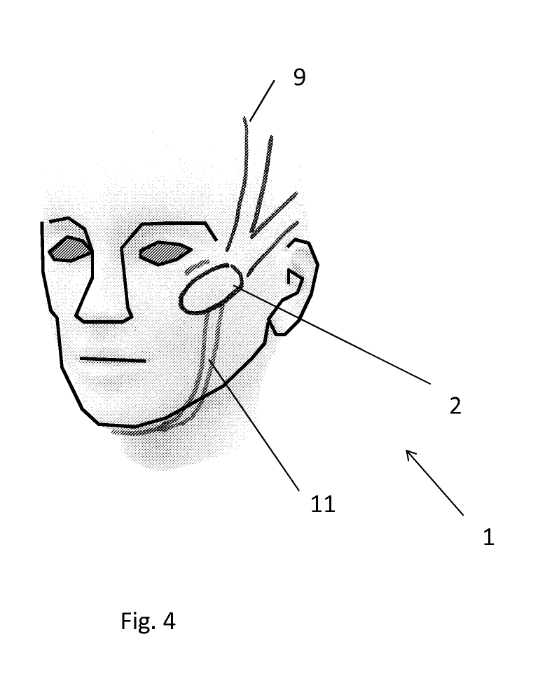

FIG. 4 illustrates a patient interface assembly 1 according to a second embodiment, when positioned on the face of a user or patient. The patient interface assembly is as depicted and described in relation with FIG. 1, but comprises further a cheek support stabilization member which is directly coupled with a first end to a first cheek mount support and with a second end to a second cheek mount support. The cheek support stabilization member is passing under and/or over the chin of the user, and can be tightened, for instance on both sides to the cheek bone stabilizer member 2. The connection can be for instance with Velcro (hook and loop fastener) on the strap or with clips. The advantage is that this stabilization strap member creates a pull down force in a direction away from the eye. Therefore the chance that the cheek bone stabilizer member 2 will slide towards the eyes is limited.

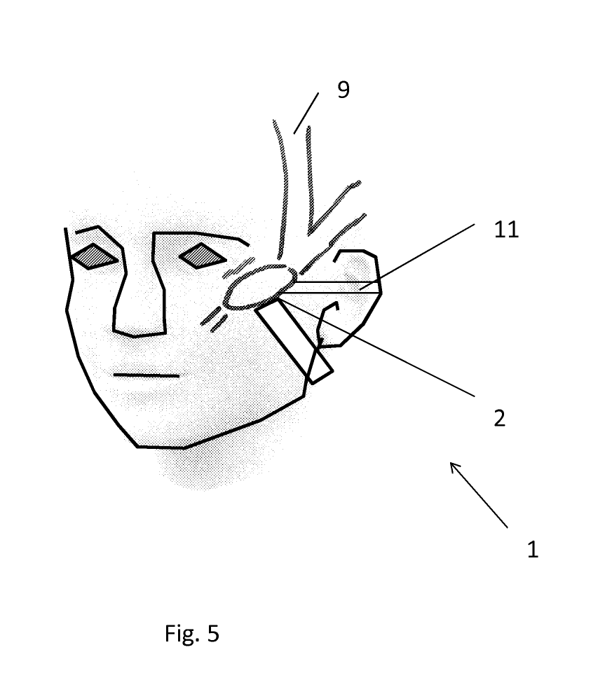

FIG. 5 illustrates a patient interface assembly 1 according to a third embodiment, when positioned on the face of a user or patient. The patient interface assembly is as depicted and described in relation with FIG. 1, but comprises further a cheek support stabilization member which is directly coupled with a first end to a first cheek mount support and with a second end to a second cheek mount support. The cheek support stabilization member is passing over the back of the head and over the ears of the user, and can be tightened, for instance on both sides to the cheek bone stabilizer member 2. The connection can be for instance with Velcro (hook and loop fastener) on the strap or with clips. The advantage is that this stabilization strap member creates a pull down force in a direction away from the eye. Therefore the chance that the cheek bone stabilizer member 2 will slide towards the eyes is limited.

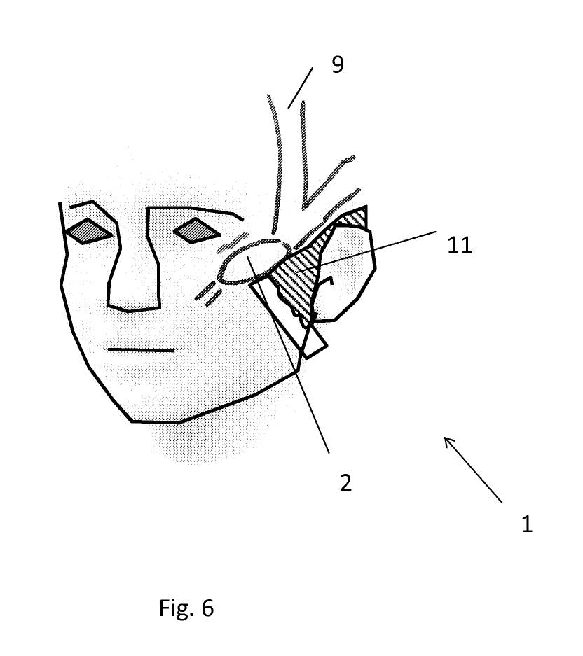

FIG. 6 illustrates a patient interface assembly 1 according to a fourth embodiment, when positioned on the face of a user or patient. The patient interface assembly is as depicted and described in relation with FIG. 1, but comprises further a cheek support stabilization member which is directly coupled with a first end to a first cheek mount support and with a second end to a second cheek mount support. The cheek support stabilization member is passing over the back of the head and around the ears of the user. Therefor the strap may comprise openings through which the ears of the user can pass when wearing the patient interface assembly. Advantageously the straps are made of a stretchable material, at least in the area corresponding to the location of the ears. It can be tightened, for instance on both sides to the cheek bone stabilizer member 2. The connection can be for instance with Velcro (hook and loop fastener) on the strap or with clips. The advantage is that this stabilization strap member creates a pull down force in a direction away from the eye. Therefore the chance that the cheek bone stabilizer member 2 will slide towards the eyes is limited.

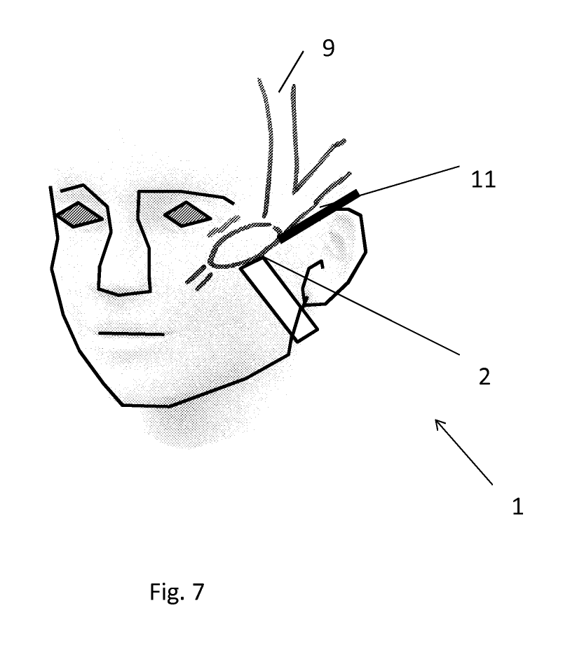

FIG. 7 illustrates a patient interface assembly 1 according to a third embodiment, when positioned on the face of a user or patient. The patient interface assembly is as depicted and described in relation with FIG. 1, but comprises further a cheek support stabilization member which is directly coupled with a first end to a first cheek mount support and with a second end to a second cheek mount support. The cheek support stabilization member is passing over the back of the head and above the ears of the user, and can be tightened, for instance on both sides to the cheek bone stabilizer member 2. The connection can be for instance with Velcro (hook and loop fastener) on the strap or with clips. The advantage is that this stabilization strap member creates a pull down force in a direction away from the eye. Therefore the chance that the cheek bone stabilizer member 2 will slide towards the eyes is limited.

The cheek support member 2 can be made from or can comprise semi-rigid or rigid material serving as a frame. For instance, materials used can be soft and comfortable, but have enough stiffness in order to allow appropriate force distribution without deforming substantially. They can comprise e.g. silicone or a silicone overmolded rigid frame, i.e. a metal or other frame being at least partially covered with a silicone layer. As the additional cheek mount support stabilization means according to embodiments of the present invention exerts forces on the cheek mount supports, the cheek mount supports are advantageously rigid.

The cheek mount support, especially when it is at least partially rigid, mainly rigid or completely rigid, can include a pad (not depicted) provided between the frame and the skin of the user, for increased comfort during wear and/or for better adhesion to the skin. This pad can be formed from any suitable material such as for instance a silicone, foam, plastic, rubber, gel or a combination thereof.

The patient interface assemblies according to any of the embodiments of the present invention can be connected to a pressure support system via a patient circuit, which communicates a gas from the pressure support system to the patient interface assembly. A patient circuit or conduit can be or comprise any device, such as a flexible tubing, that carries the flow of gas from the pressure/flow generator in the pressure support system to the patient interface assembly.

The pressure support system can be any conventional ventilation or pressure support system. Examples of such pressure support systems include, but are not limited to: a ventilator, continuous positive airway pressure (CPAP) device, or a variable pressure device, e.g. an auto-titrating device, proportional assist ventilation (PAV®) device, proportional positive airway pressure (PPAP®) device, C-Flex device, Bi-Flex device, or a BiPAP® device manufactured and distributed by Philips Respironics, Inc. of Pittsburgh, Pa., in which the pressure provided to the patient varies with the patient's respiratory cycle so that a higher pressure is delivered during inspiration than during expiration, or other pressure support device. Other devices that communicate a flow of gas with an airway of a patient suitable for use in with the present invention include devices that apply a high and low or positive and negative pressure to the airway for purposes of secretion clearance or loosening.

The patient circuit can have any suitable configuration. For example, the patient circuit can be a single-limb tubing between the pressure support system and the patient interface assembly. Alternatively, the patient circuit can be a dual-limb tubing system; having an inspiratory limb for carrying a flow of gas to the user and a expiratory limb for carrying a flow of gas from the user. Typically, a Y-connector is provided near the patient that connects the inspiratory and expiratory limbs to the patient interface assembly.

It is to be further understood that various components may be provided in or coupled to pressure support system, patient circuit, patient interface assembly, or any combination thereof. For example, a bacteria filter, pressure control valve, flow control valve, pressure/flow/temperature/humidity sensor(s), meter, pressure filter, humidifier, and/or heater can be provided in or attached to the patient circuit.

The present invention has been described with respect to particular embodiments and with reference to certain drawings but the invention is not limited thereto but only by the claims. Any reference signs in the claims shall not be construed as limiting the scope. The drawings described are only schematic and are non-limiting. In the drawings, the size of some of the elements may be exaggerated and not drawn on scale for illustrative purposes.

Where the term “comprising” is used in the present description and claims, it does not exclude other elements or steps. Where an indefinite or definite article is used when referring to a singular noun e.g. “a” or “an”, “the”, this includes a plural of that noun unless something else is specifically stated.

Furthermore, the terms first, second, third and the like in the description and in the claims, are used for distinguishing between similar elements and not necessarily for describing a sequence, either temporally, spatially, in ranking or in any other manner. It is to be understood that the terms so used are interchangeable under appropriate circumstances and that the embodiments of the invention described herein are capable of operation in other sequences than described or illustrated herein.

Moreover, the terms top, bottom, over, under and the like in the description and the claims are used for descriptive purposes and not necessarily for describing relative positions. It is to be understood that the terms so used are interchangeable under appropriate circumstances and that the embodiments of the invention described herein are capable of operation in other orientations than described or illustrated herein.

Reference throughout this specification to “one embodiment” or “an embodiment” means that a particular feature, structure or characteristic described in connection with the embodiment is included in at least one embodiment of the present invention. Thus, appearances of the phrases “in one embodiment” or “in an embodiment” in various places throughout this specification are not necessarily all referring to the same embodiment, but may. Furthermore, the particular features, structures or characteristics may be combined in any suitable manner, as would be apparent to one of ordinary skill in the art from this disclosure, in one or more embodiments.

Similarly it should be appreciated that in the description of exemplary embodiments of the invention, various features of the invention are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that the claimed invention requires more features than are expressly recited in each claim. Rather, as the following claims reflect, inventive aspects lie in less than all features of a single foregoing disclosed embodiment. Thus, the claims following the detailed description are hereby expressly incorporated into this detailed description, with each claim standing on its own as a separate embodiment of this invention.

Furthermore, while some embodiments described herein include some but not other features included in other embodiments, combinations of features of different embodiments are meant to be within the scope of the invention, and form different embodiments, as would be understood by those in the art. For example, in the following claims, any of the claimed embodiments can be used in any combination.

Furthermore, some of the embodiments are described herein as a method or combination of elements of a method that can be implemented by a processor of a computer system or by other means of carrying out the function. Thus, a processor with the necessary instructions for carrying out such a method or element of a method forms a means for carrying out the method or element of a method. Furthermore, an element described herein of an apparatus embodiment is an example of a means for carrying out the function performed by the element for the purpose of carrying out the invention.

In the description provided herein, numerous specific details are set forth. However, it is understood that embodiments of the invention may be practiced without these specific details. In other instances, well-known methods, structures and techniques have not been shown in detail in order not to obscure an understanding of this description.

Other arrangements for accomplishing the objectives of the patient interface assembly embodying the invention will be obvious for those skilled in the art. For instance other head gear arrangement, which are independent of the additional cheek mount support stabilizing member, and known to the skilled person, could be used, which, on themselves (it is without the additional cheek mount support stabilisation member) would allow the cheek mount supports only a first freedom of movement.

It is to be understood that although preferred embodiments, specific constructions and configurations, as well as materials, have been discussed herein for devices according to the present invention, various changes or modifications in form and detail may be made without departing from the scope and spirit of this invention.

1. A headgear assembly for use in a patient interface assembly adapted to communicate a flow of gas with an airway of a patient comprising:
a first and a second headgear portion disposed during use, respectively, on a first side and a second side of a patient's face in an area close to a first eye and to a second eye of said patient, wherein each of the first and the second headgear portion comprises a cheek mount support which is configured to exert a force onto opposing sides of a user's cheekbone while not exerting a force onto apex of the user's cheekbone and which comprises (i) a first member that is adapted to pass between the apex of the cheekbone and an eye of the user when the patient interface assembly is being worn by the patient and (ii) a second member that is adapted to pass below the apex of the cheekbone when the patient interface assembly is being worn by the patient;
a stabilization member (11) which is directly coupled (i) with a first end to the su port of the first headgear portion so as to limit the movement of said first headgear portion in a direction towards said first eye and (ii) with a second end to the check mount support of the second headgear portion so as to limit the movement of said second headgear portion, in a direction towards said second eye.

2. A headgear assembly according to claim 1, wherein said stabilization member is adapted and/or arranged for applying (i) a force on the first headgear portion in a direction away from the user's first eye during use and (ii) a force on the second headgear portion in a direction away from the user's second eye during use.

3. A headgear assembly according to claim 1, wherein said stabilization member is adapted and/or arranged for restricting (i) a movement of said first headgear portion such that said first headgear portion cannot move in a direction towards said user's first eye during use and (ii) a movement of said second headgear portion such that said second headgear portion cannot move in a direction towards said user's second eye during use.

4-5. (canceled)

6. A headgear assembly according claim 1, wherein said stabilization member (11) comprises a single headgear strap with at least one length adjustment means for adjusting the length of said single headgear strap.

7. A headgear assembly according to claim 6, wherein said single headgear strap comprises a first and a second length adjustment means, a first length adjustment means being adapted for being positioned at a first side of said user's face during use, and a second length adjustment means being adapted for being positioned at a second side of said user's face during use.

8. (canceled)

9. A headgear assembly according to claim 6, wherein said single headgear strap is adapted for being arranged such that it passes over the back of a head of said user and above the ears of said user when said patient interface assembly is worn by said user.

10. A headgear assembly according to claim 6, wherein said single headgear strap is adapted for being arranged such that it passes over the back of a head of said user and over the ears of said user when said patient interface assembly is worn by said user.

11. A headgear assembly according to claim 6, wherein said single headgear strap is adapted for being arranged such that it passes over the back of a head of said user and under the ears of said user when said patient interface assembly is worn by said user.

12. A headgear assembly according to claim 6, wherein said single headgear strap is adapted for being arranged such that it passes over the back of a head of said user and around the ears of said user when said patient interface assembly is worn by said user.

13. A headgear assembly according to claim 6, wherein said single headgear strap is adapted for being arranged such that it passes under and/or over a chin of said user when said patient interface assembly is worn by said user.

14. A patient interface assembly for communicating a flow of gas with an airway of a patient comprising
a headgear assembly according to claim 1,
a seal member operatively coupled to the headgear assembly, wherein the seal member is adapted to seal against a surface of said user to communicate a flow of gas with an airway of said user when using said patient interface assembly.

15. A patient interface assembly according to claim 14, wherein said seal member comprises a pair of nasal prongs, said nasal prongs being adapted for being inserted in nasal passages of said user during use.

16. A headgear assembly according claim 1, wherein said stabilization member (11) comprises two straps, (i) a first strap that is directly coupled to the cheek mount support of the first headgear portion and (ii) a second strap that is directly coupled to the cheek mount support of the second headgear portion.
